(19) United States
(12) Patent Application Publication
(10) Pub. No.: US 2026/0354922 A1
(43) Pub. Date: Nov. 12, 2026

(54) UNCONSTRAINED MASTER CONTROL DEVICE FOR A MASTER CONTROL STATION FOR MEDICAL OR SURGICAL TELEOPERATION AND CONTROL METHOD

(71) Applicant: MEDICAL MICROINSTRUMENTS, INC., Wilmington, DE (US)

(72) Inventors: Federico MONTAGNANI, Pisa (IT); Nicola PINESCHI, Pisa (IT); Massimiliano SIMI, Pisa (IT)

(21) Appl. No.: 19/477,071

(22) PCT Filed: Apr. 17, 2024
(86) PCT No.: PCT/IB2024/053737

§ 371 Date: Oct. 20, 2025

(30) Foreign Application Priority Data
Apr. 21, 2023 (IT) 102023000007839

(51) Int. Cl. A61B 34/00; A61B 34/20; A61B 34/35

(52) U.S. Cl. CPC A61B 34/74; A61B 34/35; A61B 2034/2072

(57) ABSTRACT

A master workstation for a robotic system for medical or surgical teleoperation includes at least one mechanically ungrounded master controller device with a support or handle having a surface for the palm of a surgeon's hand. A control gripper mounts to the support and includes two opposite manipulation interfaces for the fingers of the surgeon's hand. The control gripper includes a first rigid part and a second rigid part, placed side by side. The first rigid part is constrained to rotate with respect to the support about a first axis; and the second rigid part is constrained to rotate with respect to the support about a second axis, coincident with or parallel to the first axis. The tracking system is configured to detect information on position and orientation of the first rigid part and the second rigid part of the control gripper, individually.

### CROSS-REFERENCE TO RELATED APPLICATIONS

This application is a National Phase of International Application No. PCT/IB2024/053737, filed on Apr. 17, 2024, which claims priority to Italian Patent Application No. 102023000007839, filed on Apr. 21, 2023, the entire contents of which are incorporated herein by reference.

### FIELD OF THE INVENTION

The present invention relates to a master controller device.

The present invention further relates to a master workstation assembly comprising at least one master controller device.

The master controller device according to the invention is particularly suitable for a teleoperated robotic surgery system comprising a slave device operable under the control of the master controller device.

### BACKGROUND ART

Robotic surgery apparatuses are generally known in the art and typically comprise a central robotic tower or a cart and one or more robotic arms extending from the tower/cart. Each arm comprises a motorized positioning and orientation system (or manipulator) for moving a surgical instrument distally attachable thereto, in order to perform surgical procedures on a patient.

No less frequently, the “slave” surgical instrument has a distal articulation, preferably an orientation and gripping articulation, also robotically controlled by the manipulator by means of a mechanical connection interface.

The patient typically lies on an operating bed located in the operating room, in which sterility is ensured to avoid bacterial contamination due to non-sterile parts of the robotic apparatus.

In order to control the robotic manipulator and thus the slave surgical instrument, the surgeon acts on one or more master controller devices, according to a master-slave teleoperation architecture.

In known master devices, buttons are typically provided to transmit control signals to the slave surgical instrument.

Other known master controller devices include articulated structures to support the manipulated part, which are adapted to record or detect movements in space (translations) of such a gripped and manipulated part in space, as shown for example in U.S. Pat. No. 5,808,665 and WO-2020-188390.

In some prior art examples, the right and left master devices are each provided to be mounted to an attachment of the operative console and supported by a gimbal system adapted to detect the orientation inputs.

Otherwise, master devices of the type mechanically/kinematically ungrounded to the operative console (“groundless” or “unconstrained” or “UID” according to the jargon adopted in this field) are known, i.e., of the “steering wheel-type”, which is manipulated by the surgeon in a predeterminable three-dimensional tracking volume. Such ungrounded “steering wheel” master devices can be suitable for monolateral teleoperation without force feedback.

As shown for example in the prior art document WO-2019-220407 to the same Applicant, an elastic element can be provided, specifically a pre-loaded trigger in a cantilevered position between two rigid rods, which is capable of simulating a sort of feedback to the surgeon, stiffening the resistance to closing of the rods, when the opening/closing angle between the rods of the master device is less than a certain predeterminable threshold. A similar solution, showing a master device with differentiated stiffness during closure, is disclosed by U.S. Pat. No. 6,594,552.

Ungrounded master devices are usually equipped with sensors, such as inertial platforms and/or position and/or orientation sensors, such as magnetometers and/or optical markers, to cause the command to be transmitted to the slave surgical instrument. In some known examples, a magnetic field emitter is provided, which generates a tracking volume in which the position and orientation of two magnetometer-type sensors with six degrees of freedom provided on the body of the master device are tracked, so as to provide closing command signals to the slave device when the detected distance between the sensors is less than a certain threshold.

For example, document WO-2022-175800 to the same Applicant shows an ungrounded master device solution in which the system control unit verifies the existence of predefined geometric relationships between two sensors integrated the master device itself.

For example, WO-2022-175792 to the same Applicant shows a solution in which the slave surgical instrument is uniquely identified by means of a virtual control point which is halfway between the two tips (or“ jaws”) of the surgical instrument, which in the master workspace can coincide with the midpoint between the sensors mounted to the rods of the body of the master device.

For example, WO-2022-175802 to the same Applicant shows a safety system solution for an ungrounded master device, designed to disable teleoperation in the event of excessive accelerations/speeds detected by the integrated sensors.

The known solutions of a master device not constrained to the operative console mentioned above, although partially advantageous in some respects, are not at all without drawbacks.

In fact, the known ungrounded master controller devices could slip, slide, bump into objects or even fall out of the hand during manipulation, transferring and causing unexpected and unwanted movements of the end-effector of the teleoperated system and thus on the patient.

Once grasped, the ungrounded master controller devices could be uncomfortable in manipulation or result in a limited precision mobility of the degrees of freedom thereof during the manipulation thereof.

WO-2019-099584 and US-2020-0390510 disclose some further examples of master devices of the ungrounded type, and in particular a solution which includes a sort of handle or grip wearable by the surgeon, equipped with special rings for the surgeon's fingers, which can be connected to the opening/closing command operating portion also by means of a spherical joint. A similar solution is disclosed in US-2020-0237467 in which the wearable portion of the master device is a sort of bracelet from which an articulated connection extends to the sensorized portion of the master controller device, in which a tracking sensor and buttons for activating the open/close command are included.

Although solving some aspects of instability, the embodiments of the master device presented have a high number of mechanical and electronic components, a high construction complexity, the difficulty of a form which can be sterilized or draped for sterile manipulation, and lastly kinematics which are too complex to limit the simple and free manipulation of the natural degrees of freedom of the hand compromising control, precision, accuracy and range-of-motion.

The need is thus strongly to suggest a master device solution with improved ergonomics and safety with respect to known solutions, without resulting in a worsened master-slave teleoperation accuracy or limiting the relative range-of-motion or sterility.

At the same time, the need is felt to provide an intuitive and ergonomic master device to be used which is capable of guaranteeing a satisfactory reliability in the control of a plurality of degrees of freedom of the slave device, which include the open/close degree of freedom i.e., gripping and/or cutting of the surgical instrument of the slave device.

### SOLUTION

It is an object of the present invention to obviate the drawbacks complained of with reference to the prior art.

According to an aspect of the invention, a master workstation assembly for a robotic system for medical or surgical teleoperation comprises at least one mechanically ungrounded master controller device, adapted to control at least one open/close degree of freedom and one yaw degree of freedom of a slave surgical instrument of the robotic system, and a tracking system for detecting position and orientation information of the master controller device.

The ungrounded master controller device comprises a support or handle comprising a surface for the palm of a surgeon's hand, and a control gripper mounted to the support and comprising two manipulation interfaces for the fingers of said surgeon's hand.

The control gripper comprises a first rigid part and a second rigid part placed side by side, said first rigid part being constrained to rotate with respect to the support about a first axis, said second rigid part being constrained to rotate with respect to the support about a second axis, coincident with or parallel to said first axis, and the tracking system is configured to detect information on position and orientation of the first rigid part and the second rigid part of the control gripper, individually.

The angular stroke of the first rigid part is preferably superimposed on the angular stroke of the second rigid part and in accordance with an embodiment the respective angular strokes of the two rigid parts of the control gripper are substantially coincident.

The tracking information on position and orientation of the master controller device is preferably acquired by means of two identifiers (for example sensors) with six degrees of freedom arranged one per rigid part, as well as the opening/closing information between the rigid parts.

Therefore, the control gripper is a sensorized control gripper for detecting at least the open/close and yaw degrees of freedom. Therefore, the handle or support can be transparent to the tracking system, acting as an ergonomic component to impose a certain positioning of the surgeon's hand and fingers on the master controller device, so as to operate the slave degrees of freedom of at least open/close and yaw.

It therefore allows the open/close and yaw manipulation of the slave device with the fingers (for example index and thumb) of a surgeon's hand, allowing a finer control on the micro-movements to be carried out with respect to the use, for the surgeon, of the wrist or elbow for the activation of the yaw joint of the slave surgical instrument controlled by the master controller device. The provision of the support or handle comprising a surface for the palm of a surgeon's hand imposes on the surgeon a certain positioning of the fingered hand on the sensorized control gripper.

A further translational and/or rotational joint can be provided, arranged between the control gripper and the support or handle, to allow the manipulation, with the same fingers of the surgeon, of other degrees of freedom of the slave device.

An elastic element can be included for biasing the further joint toward a predetermined position.

### BRIEF DESCRIPTION OF THE DRAWINGS

Further features and advantages of the invention will become apparent from the following description of preferred embodiments, given by way of non-limiting indication, with reference to the accompanying drawings which are briefly described below. Note that references to “an” embodiment in this disclosure do not necessarily refer to the same embodiment, and are to be understood as at least one. Moreover, for reasons of conciseness and reduction of the total number of figures, a certain figure can be used to illustrate the features of more than one embodiment, and not all the elements of the figure can be necessary for a certain embodiment.

FIG. 1 shows an axonometric view of a robotic system for medical or surgical teleoperation, according to an embodiment.

FIG. 2 shows an axonometric view of a slave surgical instrument, according to an embodiment.

FIGS. 3 A, 3 B, 3 C and 3 D show a pictorial perspective view of a master controller device, according to an embodiment, held in hand by a surgeon in open configuration and in closed configuration.

FIG. 4 diagrammatically shows a tracking device of the master workstation assembly, according to an embodiment.

FIGS. 5 and 6 diagrammatically show a tracking device of the master workstation assembly, according to some embodiments

FIG. 7 A, 7 B, 7 C, 7 D, 7 E and 7 F diagrammatically show some possible configurations of the control gripper of the master controller device and of the control point, according to an embodiment.

FIG. 8 diagrammatically shows a master controller device, according to an embodiment.

FIGS. 9 A and 9 B show an axonometric view and a top plan view, respectively, of a master device comprising a joint between support or handle and control gripper, in accordance with an embodiment, in which some parts are shown in section for clarity.

FIG. 10 A shows an axonometric view of a master controller device, according to an embodiment.

FIG. 10 B is a longitudinal sectional view of the master controller device of FIG. 10 A.

FIG. 10 C shows a sectional view of the joint between support or handle and control gripper of the master controller device of FIG. 10 A.

FIG. 11 pictorially shows a master controller device, according to an embodiment, when held in hand by a surgeon.

FIGS. 12 A and 12 B show a pictorial vertical elevation view of some possible configurations of a handle or support of a master controller device, according to some embodiments.

FIGS. 13 A and 13 B are diagrams showing a master controller device comprising a joint between the control gripper and the handle or support, according to an embodiment, in some operating configurations.

FIG. 14 shows a longitudinal sectional view of a master controller device, according to an embodiment.

FIGS. 15 A and 15 B show a sectional view of a master controller device, according to some embodiments.

FIG. 16 shows an axonometric view of a master controller device and a reference of a master workstation, according to an embodiment.

FIG. 17 shows a pictorial axonometric view of a master controller device, according to an embodiment.

FIG. 18 pictorially shows a master controller device, according to an embodiment, which is held in hand by a surgeon.

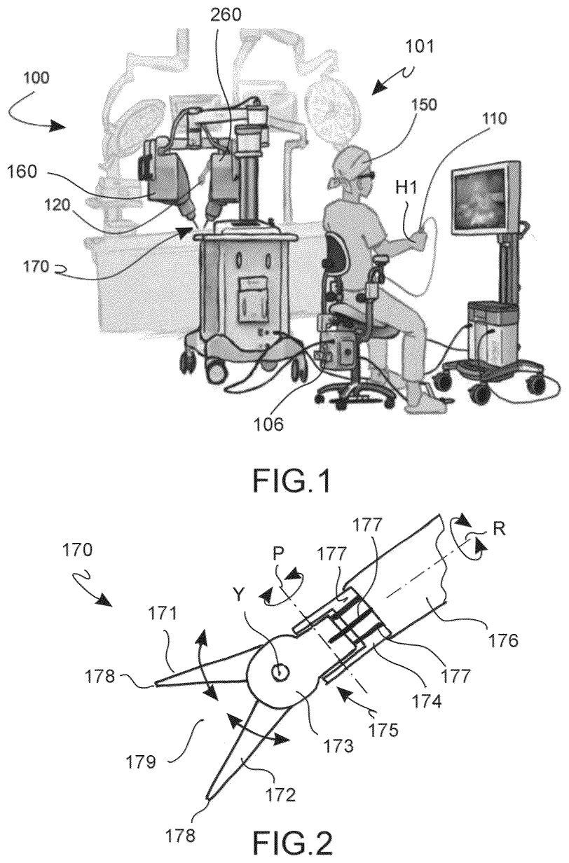

### DETAILED DESCRIPTION OF SOME EMBODIMENTS

Reference throughout this description to “an embodiment” means that a particular feature, structure or function described in relation to the embodiment is included in at least one embodiment of the present invention. Therefore, the formulation “in an embodiment” in various parts of this description do not necessarily all refer to the same embodiment. Moreover, particular features, structures or functions such as those shown in different drawings can be combined in any suitable manner in one or more embodiments.

In accordance with a general embodiment, a master workstation assembly 101 (or master workstation 101) is provided.

The master workstation assembly 101 is particularly suitable for a robotic system 100 for medical or surgical teleoperation.

The robotic system 100 further comprises at least one slave surgical instrument 170 movable under the control of the master controller device 110 according to a master-slave teleoperation architecture.

The slave surgical instrument 170 comprises an articulated end comprising at least one rotational joint between a support link 173 and two respective tip links 171, 172 (or jaws 171, 172) for moving the tip links 171, 172 with respect to the support, preferably about a common rotation axis Y, or yaw rotation axis Y of the slave surgical instrument 170. The tip links or jaws preferably have a free end and a root for attaching to an articulation pin defining the yaw rotation axis Y of the articulated end of the slave surgical instrument.

The articulated end 175 of the slave surgical instrument 170 can further comprise a proximal link 174 which is articulated to the support link 173 and arranged proximally with respect thereto, to move the support link 173 about a pitch rotation axis P. The articulated end of the slave surgical instrument 170 can further comprise a roll degree of freedom, for example to allow the rotation of the articulated end about a longitudinal axis R, or roll axis R, which preferably extends along a positioning rod or shaft 176 of the slave surgical instrument 170.

Preferably, the links 171, 172, 173 of the articulated end 175 of the slave device are moved by means of actuation tendons 177, for example two actuation antagonist tendons are included for each link.

In accordance with a preferred embodiment, each tip link 171, 172 or jaw 171, 172 is individually moved by means of the inclusion of two actuation tendons thereof with antagonistic effects. In other words, each tip link 171, 172 is articulated to the support link 173 in the yaw rotation axis Y and is individually moved. Thereby, it is possible to obtain both the open/close degree of freedom (for example gripping or cutting) and the yaw degree of freedom of the articulated end 175 of the slave surgical instrument 170. In other words, yaw and open/close degrees of freedom of the slave surgical instrument 170 are obtained by rotational joints of the articulated end 175 of the slave surgical instrument 170 which are preferably actuated by means of actuation tendons.

The master workstation assembly 101 comprises at least one master controller device 110, of the type mechanically ungrounded to the operative console, and at least one tracking device for detecting information on position and orientation of the master controller device 110 within a tracking volume.

The master controller device 110 is of the type mechanically not constrained to the operative console, i.e., of the type not connected to the ground (“ungrounded”, “UID”, “movable in the free working space”) and preferably is a master controller device 110 of the steering wheel type without force feedback.

The master controller device 110 is adapted to control at least one open/close degree of freedom and at least one yaw degree of freedom of said slave surgical instrument 170 of the robotic system. In other words, by means of the ungrounded master controller device 110, the movement of at least the two tip links 171, 172 with respect to the support link 173 of the articulated end 175 of the slave surgical instrument 170 is controlled.

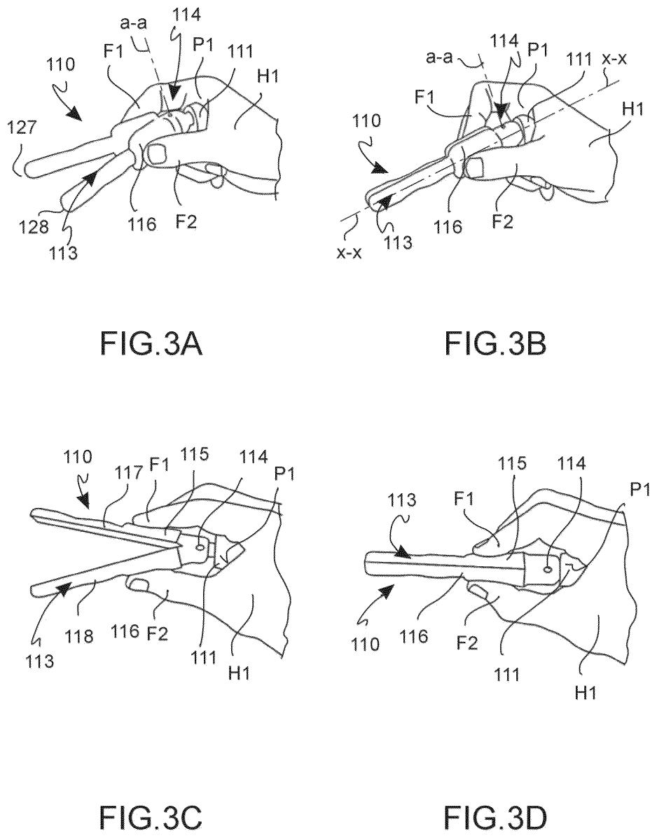

As shown for example in FIG. 3 A-D, the master controller device 110 comprises a handle 111 or support 111 comprising a surface 112 for the palm P1 of a hand H1 of a surgeon 150 and a control gripper 113 mounted to the support 111 and comprising two opposite manipulation interfaces 115, 116 for the fingers F1, F2 of said hand H of the surgeon 150. The manipulation interfaces 115, 116 are arranged opposite with respect to a definable longitudinal axis X-X of the control gripper so that the action of the surgeon's fingers (for example index F1 and thumb F2 of the hand H) is aimed at closing the control gripper toward the definable longitudinal axis.

In accordance with a preferred embodiment, said support 111 is a handle 111 forming the grip of the master controller device 110. The body of the handle or support can be shaped, for example obtaining it by molding or by additive manufacturing (for example 3D printing) to provide ergonomic features for the surgeon's hand so as to optimize the grip on the control gripper.

Advantageously, the control gripper 113 comprises a first rigid part 117 and a second rigid part 118. Said first rigid part 117 is constrained to rotate with respect to the support 111 about a first axis a-a and said second rigid part 118 is constrained to rotate with respect to the support 111 about a second axis a′-a′, which is coincident with or parallel to said first axis a-a. Therefore, where said first axis a-a and said second axis a′-a′ are coincident, the first rigid part and the second rigid part of the control gripper 113 are constrained to rotate with respect to the support 111 about a common axis a-a. The relative approach/distancing movement between the rigid parts of the control gripper preferably occurs in a predefined plane which is orthogonal to the axis a-a, a′-a′.

The rigid parts 117, 118 preferably have a rigid and elongated body. The rigid parts 117, 118 of the control gripper 113 are preferably rigid rods or fins each having a free end 127, 128 and a root for attaching to the joint 114, 114′. The rigid parts 117, 118 preferably extend substantially straight, i.e., straight away from the handle and mutually diverging because they are influenced in opening by an elastic element 126.

The control gripper 113 preferably does not comprise any sensorized body between the rigid parts 117, 118 which can therefore sweep a wide angular amount, and in accordance with a preferred embodiment, the rigid parts 117, 118 are capable of sweeping a substantially coincident or superimposed angular amount, i.e., they are movable independently or jointly in a same angular range. For example, therefore, the two rigid parts can be oriented with respect to the handle within an angular stroke β which sweeps 45° to the right of the handle and 45° to the left of the handle, i.e., with respect to a centered reference position, when in operating conditions, i.e., when the handle is gripped by the hand H of the surgeon 150 with index finger F1 and thumb F2 on the interface portions 115, 116 of the rigid parts 117, 118 of the control gripper.

With further advantage, said tracking system is configured to detect information on position and orientation of the first rigid part 117 and the second rigid part 118 of the control gripper 113, individually. In other words, the master workstation assembly 101 detects information on position and orientation of each rigid part 117 or 118 of the control gripper 113 of the master controller device 110.

Therefore, the control gripper is the portion which is sensorized and used to control the slave surgical instrument while the handle or support has an ergonomic function, although in accordance with an embodiment the handle or support is fitted with one or more buttons or other commands for operating a functionality of the slave device such as, for example, changing the master-slave scale factor. Therefore, the handle or support is transparent to the tracking system while the control gripper is sensorized, i.e., it is read by the tracking system by virtue of the provision of appropriate identifiers arranged on the rigid parts of the control gripper.

In accordance with a preferred embodiment, said tracking system comprises a reference 106 defining a master reference system MFO and two identifiers 107, 108 fixed to the respective rigid parts 117, 118 of the control gripper 113, to detect information on position and orientation of each rigid part 117 or 118 with respect to the reference 106. For example, each identifier 107, 108 defines a local reference system MF1, MF2 which is fixed with respect to the respective rigid part 107 or 108 of the control gripper 113.

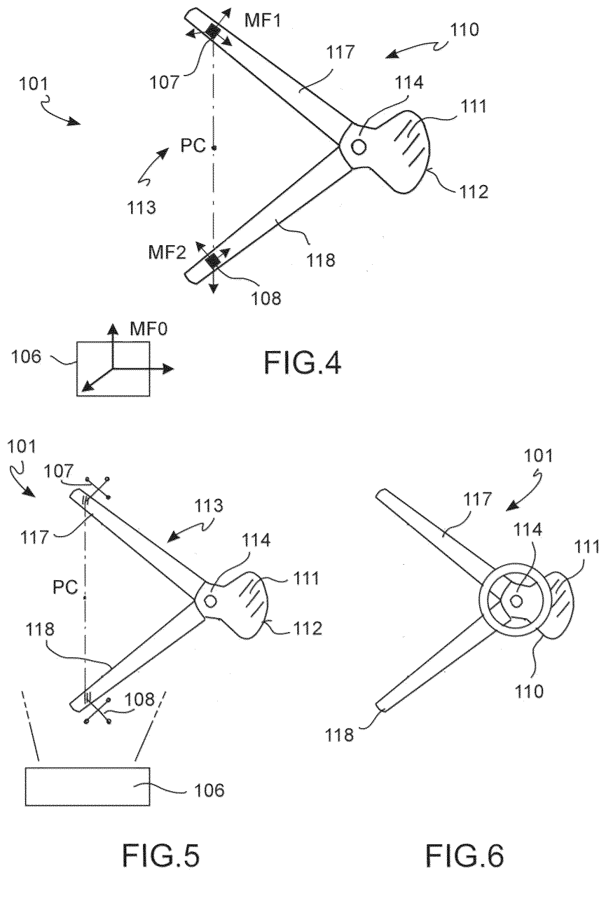

As shown for example in FIG. 4, in accordance with a preferred embodiment, the tracking system comprises two tracking sensors 107, 108, for example magnetometer type sensors, each fixed to a respective rigid part 117, 118 of the control gripper 113, acting as identifiers, and a tracking field generator 106, for example a magnetic field generator, acting as said reference.

As shown for example in FIG. 5, the tracking system can comprise two optical markers 107, 108, each fixed to a respective rigid part 107, 108 of the control gripper 113, acting as identifiers, and an optical detector 106, for example a camera, acting as a reference. The identifiers 107, 108 can be the rigid parts 117, 118 themselves or portions thereof, such as a distal portion thereof with free end.

As shown for example in FIG. 6, the tracking system can have two encoders mounted to the support 111 and each operatively associated with a respective rigid part 117, 118 of the control gripper 113. The identifiers 107, 108 can be the rigid parts 117, 118 themselves or portions thereof, such as a distal portion thereof with free end.

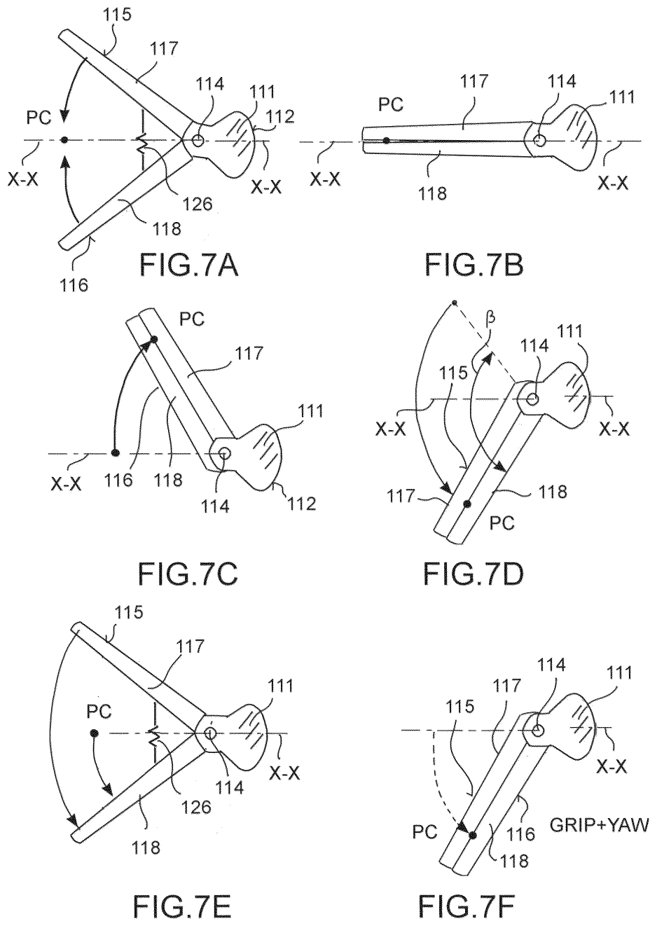

In accordance with a preferred embodiment, the control gripper 113 comprises an elastic element 126 (for example diagrammatically shown in FIG. 7 A) between the first rigid part 117 and the second rigid part 118 biasing the first rigid part 117 and the second rigid part 118 away from each other. The elastic element of the control gripper 113 can comprise a torsional spring mounted to a rotational joint, for example of the pin type, between the rigid parts 117, 118. The elastic element of the control gripper 113 can comprise an axial spring, for example helical, arranged between the rigid parts 117, 118 of the control gripper 113.

The inclusion of the elastic element biasing the first rigid part 117 and the second rigid part 118 away from each other requires the surgeon 150 to close the control gripper 113 bringing the manipulation interface portions 115, 116 and thus the rigid parts 117, 118 of the control gripper closer together, counteracting the elastic biasing action.

The inclusion of the elastic element 126 biasing the first rigid part 117 and the second rigid part 118 away from each other allows determining a rest configuration of the control gripper 113 having a certain distance and/or angle between said identifiers 107, 108.

The tracking system can be configured to calculate, based on the detected information on position and orientation of the first rigid part 117 and the second rigid part 118 of the control gripper 113 individually, a control point PC (a virtual point rigidly associated with the control gripper) containing information on position and orientation of the control gripper 113. Preferably, said control point PC is calculated as the midpoint between the rigid parts of the control gripper, i.e., as the midpoint between the detected positions of the identifiers 107, 108. In other words, the control point PC can contain information for controlling the yaw degree of freedom Y of the slave surgical instrument 170, i.e., the overall orientation of the tip links 171, 172 or jaws 171, 172 of the articulated end 175 with respect to the support link 173. Therefore, the control point PC between the rigid parts 117, 118 can be a virtual point which does not belong to a part of the body of the master device, since no sensorized element is provided between the two rigid parts to allow the superimposed angular stroke β of the rigid parts for controlling the yaw degree of freedom Y of the slave surgical instrument 170.

The slave surgical instrument 170 can in turn be controlled by defining a slave control point 179 (a virtual point rigidly associated with the articulated end 175), for example, the midpoint between the tips or jaws 171, 172, containing information on position and orientation of the articulated end 175 (for example, 6 degrees of freedom of orientation and position and preferably information on opening and closing force).

The information on the degree of opening/closing between the rigid parts 117, 118 of the control gripper 113 can be obtained by detecting the distance between the identifiers 107, 108 and/or the angle between the identifiers 107, 108 and added to the information contained in the control point PC.

In accordance with an embodiment, each rigid part 117, 118 of the control gripper 113 has an angular stroke β of about 90°, centered on a reference position, which for example provides for the rigid part 117 or 118 substantially aligned with a definable longitudinal axis of the control gripper 113. In other words, each rigid part 117, 118 is movable within a range from −45° to +45° with respect to a definable reference position.

The reference position of the first rigid portion 118 can be aligned and substantially coincident with the reference position of the second rigid portion 118.

Preferably, no tracking sensor or identifier is provided in the handle 111, and the activation of the slave yaw degree of freedom (rotational joint) is caused by the position taken by the surgeon while manipulating the master controller device by gripping it from the handle 111 and maneuvering the sensorized control gripper with his fingers F1, F2. In other words, the handle 111 is transparent to the tracking system because it has no tracking sensors nor identifiers.

The angular stroke β of the first rigid part 117 can be superimposed on the angular stroke β of the second rigid part 118, and preferably coincident therewith.

Therefore, the control point PC calculated by the tracking device of the master workstation assembly 101 can have an angular stroke β belonging to the range from −45° to +45° with respect to a definable reference position. The reference position of the control point PC is preferably aligned with a definable longitudinal axis X-X of the control gripper.

As shown for example in FIG. 7 A-B, by bringing the rigid parts 117, 118 of the control gripper 113 substantially symmetrically together, i.e., at the same speed, the position of the control point PC remains aligned with a definable longitudinal axis X-X of the control gripper 113.

As shown for example in FIG. 7 C-D, in closed (or semi-closed) control gripper 113 conditions, in which the rigid parts 117, 118 are abutting against each other, it is possible to move the control point PC, jointly rotating both rigid parts 117, 118. Thereby, a control is made on the joint orientation of the tip links 171, 172 or jaws 171, 172 of the slave device 170 with respect to the support link 173, while the tip links 171, 172 remain in closed or semi-closed configuration (for example, in gripping or cutting conditions). In other words, the yaw rotational joint of the articulated end 175 of the slave surgical instrument 170 is allowed to operate.

As shown for example in FIG. 7 E-F, in open control gripper 113 conditions, it is possible to move the control point PC by moving a single rigid part 117 or 118 in its angular stroke with respect to the support 111 and keeping the other rigid part 118 or 117 stopped or substantially stopped, with respect to the support 111.

By virtue of such a solution, it is possible to obtain a transmission of commands from master to slave, through the definition of a control point PC which controls the slave control point 179.

The inclusion of said rotational joint 114 between the rigid parts of the control gripper 113 allows moving the control point PC with respect to the handle 111 or support 111 in a plane defined between the identifiers 107, 108 of the rigid parts 117, 118 to which the yaw degree of freedom of the master controller device 110 belongs, which is therefore controlled with the surgeon's fingers (index finger and thumb, for example), avoiding controlling the yaw positioning (yaw) with the surgeon's wrist/elbow. Accordingly, the yaw degree of freedom Y of the articulated end 175 of the slave surgical instrument 170 is controlled by means of the surgeon's fingers.

The robotic system 100 is preferably configured so that, even if the reference 106 for the identifiers 107, 108 is not located on the handle 111 or support 111, the movement therebetween, i.e., the movement between the rigid parts 117, 118 of the control gripper 113 about the axis a-a due to the joint 114 only activates the yaw degree of freedom Y of the articulated end 175 of the slave surgical instrument 170. This is because the orientation of the handle (support) and/or the control gripper in roll and pitch is transmitted to the rigid parts 117, 118 and thus to the identifiers 107, 108, by rotating the control point PC and then transmitting roll rotation and pitch to the slave control point 179 which activates the relative roll R and pitch P joints of the articulated end 175.

In accordance with an embodiment, the control gripper 113 is rigidly fastened to the handle 111 or support 111, so that the control gripper 113 is not re-orientable with respect to the handle 111. Thereby, roll and pitch rotations of the handle or support involve the rotation of the control point PC and are transmitted to the roll and pitch joints of the slave device 170.

The translations imposed on the control point PC can be managed by translating the positioning shaft or rod 176 of the slave surgical instrument 170.

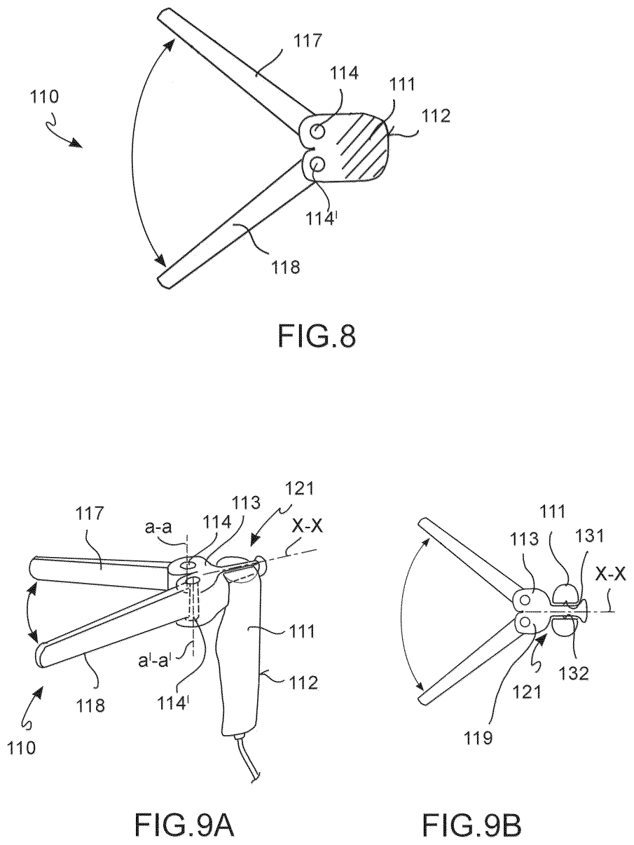

In accordance with an embodiment, as shown for example in FIG. 8, each rigid part 117, 118 of the control gripper 113 is fastened at a rotational joint 114, 114′ thereof to the support 111, individually. The rotation axes of each rigid part 117, 118 with respect to the support 111 are parallel to each other. Thereby, the two rigid parts are each hinged to the handle in an independent rotational joint 114, 114′, and each such as to cause relative movement with respect to the handle as well as with respect to the other rigid part. An elastic element can be included for biasing the rigid portions 117, 118 away relative to each rotational joint 114, 114′. Alternatively or in addition, an elastic element can be provided between the two rigid parts 117, 118.

The two rotational joints can be arranged coaxial. In accordance with an embodiment, the control gripper 113 comprises a rotational joint 114 thereof between the two rigid parts 117, 118, constraining them to rotate about a common axis which coincides with said first axis. Thereby, the two rigid parts are hinged to the handle in a single rotational joint 114 such as to cause relative movement with respect to the handle as well as of one rigid part with respect to the other. An elastic element 126 can be provided to bias the rigid parts 117, 118 away relative to the common rotational joint 114. Alternatively or in addition, an elastic element 126 can be provided between the two rigid parts 117, 118.

Preferably, said at least one rotational joint 114, 114′ is a cylindrical joint.

In accordance with a preferred embodiment, the master controller device 110 comprises a joint between the support 111 and the control gripper 113. In other words, there is a further degree of freedom of the control gripper 113 with respect to the support 111 or handle 111, in addition to the open/close degree of freedom and the yaw degree of freedom provided by the at least one rotational joint 114, 114′ between the rigid parts 117, 118 of the control gripper 113. Thereby, said first and second rigid parts 117, 118 of the control gripper 113 can be connected in an opening/closing and yaw joint 114, 114′ to an intermediate mechanical link in turn having a relative motion (or degree of freedom) with respect to the handle or support of the master controller device.

In accordance with an embodiment, as shown for example in FIG. 9 A-B, the master controller device 110 comprises, between the support 111 and the control gripper 113, a cylindrical rotational joint 121 with an axis substantially parallel to the longitudinal axis X-X of the control gripper 113. This allows a roll degree of freedom of the control gripper 113 with respect to the handle 111 or support 111. It is thus also possible to control with the fingers F1, F2 of the hand H of the surgeon 150, instead of with the surgeon's wrist/elbow, the roll degree of freedom of the master controller device 110 and therefore the roll or rotation degree of freedom about the axis R of the slave surgical instrument 170. For example, the handle 111 can comprise a longitudinal hole or cavity having a cylindrical inner wall 131 engaging a cylindrical counter-surface 132 of the control gripper 113. The control gripper 113 can comprise a hub 119 to which both rigid parts 117, 118 are rotatably connected by means of said one or more rotational joints 114, 114′, said hub 119 of the control gripper 113 comprising a longitudinal tail comprising said cylindrical surface 132 which is received in the cylindrical hole 132 of the handle 111.

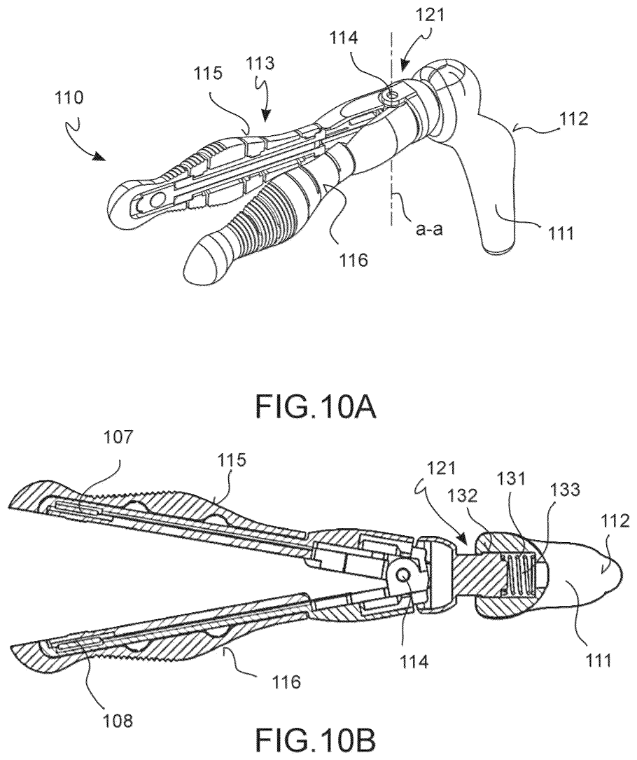

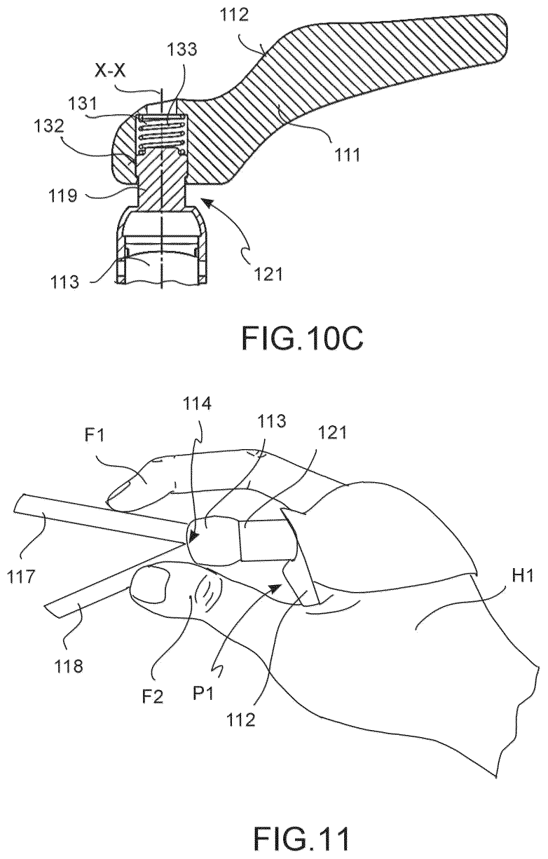

In accordance with an embodiment, as shown for example in FIG. 10 A-C, the master controller device 110 comprises, between the support 111 and the control gripper 113, a cylindrical rotational and translational joint 121. In other words, the joint also provides, in addition to a cylindrical rotational joint with an axis substantially parallel to the longitudinal axis X-X, a translation degree of freedom of the control gripper 113 along the longitudinal direction X-X, and for example can allow the retraction of the control gripper 113 with respect to the support 111. Therefore, the joint can comprise a longitudinal hole or cavity having a cylindrical inner wall 131 engaging with a cylindrical counter-surface 132 of the control gripper 113, the hole or cavity having a longitudinal extension thereof such as to guide with the cylindrical inner wall 131 thereof the movement in the longitudinal direction of the control gripper 113. A spring 133 can be included for biasing the control gripper 113 in advancement with respect to the support 111. When in operating conditions, by moving the rigid parts 117, 118 closed with the fingers F1, F2 of the hand H of the surgeon 150, the kinematics of the human hand imposes a not perfectly circular trajectory which has a physiological retraction, which can be favorably accommodated by the inclusion of said longitudinal translation degree of freedom of the joint between the handle and the control gripper.

The spring 133 can have a self-centering effect on the control gripper 113, biasing the joint 121 towards the resting position thereof. In other words, the rotational joint between handle and control gripper can comprise a spring (not shown) for biasing the joint towards a predetermined angular position (orientation).

The retraction/advancement translation allowed by the joint allows controlling, if necessary, with the fingers F1, F2 of the hand H gripping the support 111 also a translation degree of freedom of the slave surgical instrument 170. For example, the robotic manipulator 160 can comprise one or more linear sliding guides to cause the retraction/advancement translation of the positioning shaft or rod 176 of the slave surgical instrument 170., The tracking system and/or an associable control system can inhibit the translation of the slave surgical instrument 170 in response to this rearward accommodation of the control gripper 113 with respect to the handle 111.

As shown for example in FIG. 11, the support 111 can be a wearable element comprising an inner surface 112 for the palm P1 of the surgeon's hand H 150 and two opposite interface portions 115, 116 on the control gripper 113 for manipulation by the surgeon's fingers F1, F2. In this embodiment, a roll rotational joint 121 is provided between the support 111 and the control gripper 113, allowing the surgeon 150 to maneuver with the fingers F1, F2 of his or her hand H wearing the support 111 the open/close, yaw and roll degrees of freedom of the master controller device 110, which translate into controlling the yaw and roll joints of the slave surgical instrument 170.

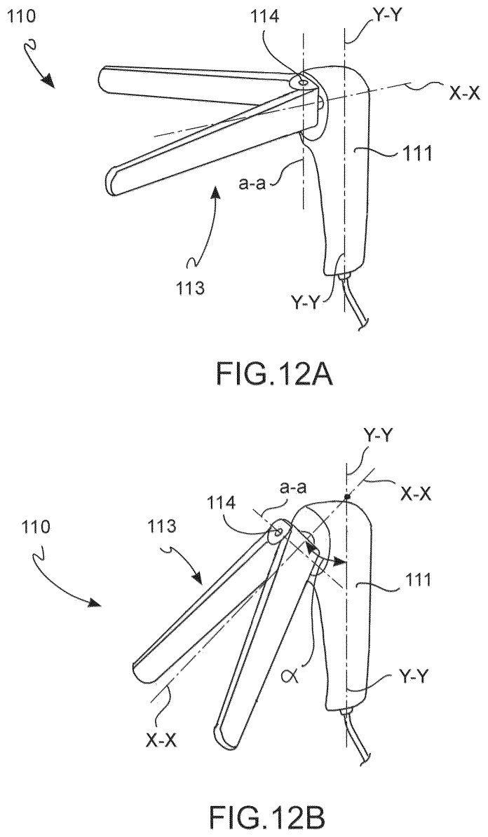

As shown for example in FIG. 12 A, the body of the handle 111 can have an extension thereof along an extension direction Y-Y thereof which can be substantially parallel to the rotation axis between the two rigid parts 117, 118 determined by the at least one joint 114, 114′.

As shown for example in FIG. 12 B, the extension direction Y-Y of the handle 111 can form an acute pitch angle a with the longitudinal direction X-X of the control gripper 113. Preferably, the handle 111 is rigid and the angle between the extension direction Y-Y thereof and the longitudinal direction X-X of the control gripper 113 is rigidly predeterminable.

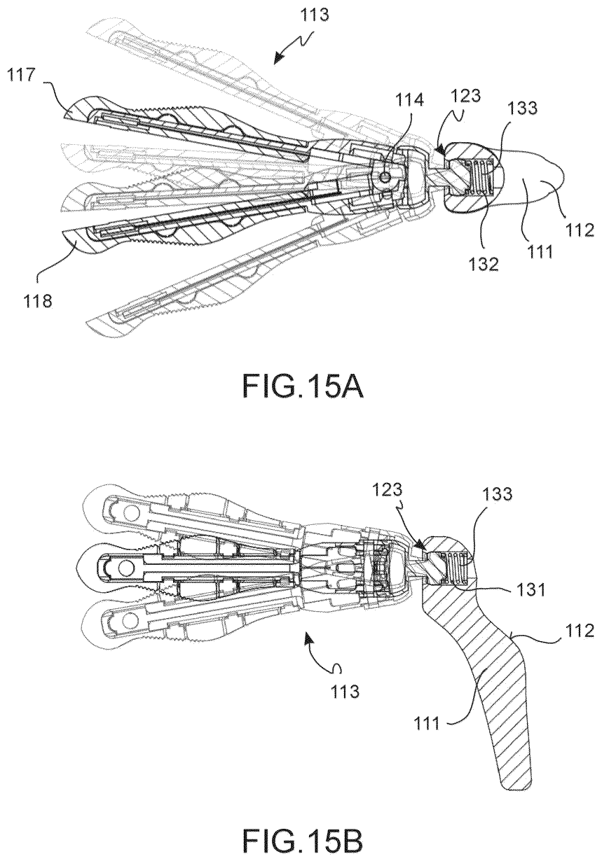

In accordance with an embodiment, the joint between handle 111 or support 111 and control gripper 113 also allows orienting the control gripper in pitch, as shown for example in FIG. 15B, and for example said joint comprises a spherical joint 123.

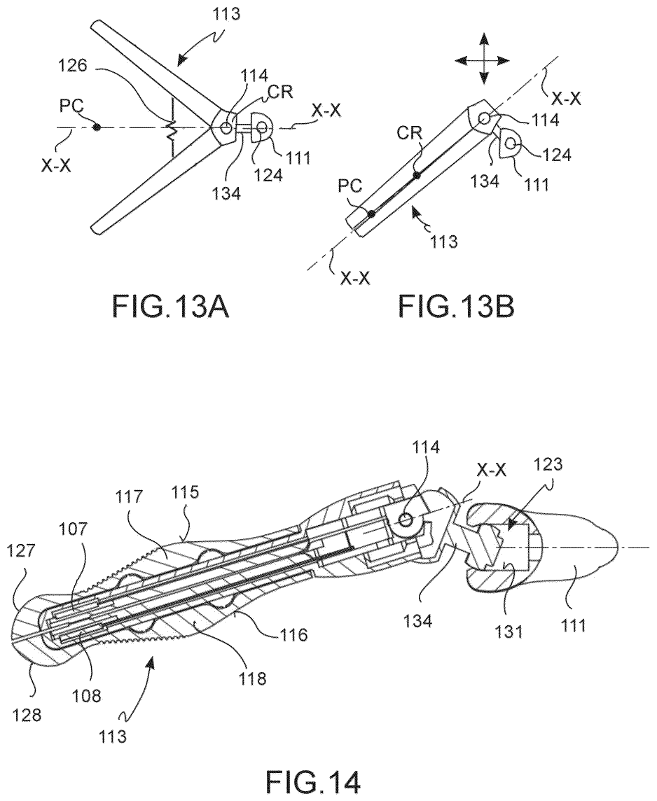

As shown for example in FIG. 13 A-B, the joint between support 111 and control gripper 113 can comprise a rotational joint 124 which allows the rotation about an axis substantially parallel to the at least one rotation axis a-a, a′-a′ of the at least one rotational joint 114, 114′ between the rigid parts 117, 118 of the control gripper 113. In accordance with an embodiment, said rotational joint 124 is a cylindrical joint. Between the cylindrical joint 124 and the at least one rotational joint 114, 114′ of the control gripper 113, a connecting rod 134 and/or a section 134 of rigid body can be provided, which allows widening the yaw orientation range-of-motion of the control gripper 113, by moving the instantaneous rotation center CR of the control gripper with respect to the support 111 in a distal direction with respect to the at least one joint 114, 114′, i.e., toward the control point PC, thereby bringing the instantaneous rotation center CR of the control gripper 113 closer to the manipulation interface portions 115, 116 on which the surgeon 150 rests his fingers F1, F2 to maneuver the control gripper 113. It is therefore possible, by virtue of said cylindrical joint 124 parallel to the joint 114 between the rigid parts 117, 118, between the control gripper and the handle, to move the joint 114 in a plane by means of manipulation with the fingers F1, F2.

In accordance with a preferred embodiment, as shown for example in FIG. 14, between the support 111 or handle 111 and the control gripper 113 there is a joint comprising a spherical joint 123 and a linear guide 131 or cavity 131 for the longitudinal movement of the spherical joint 123 and therefore of the control gripper 113. Said spherical joint 123 can be associated with a spring 133, as shown for example in FIG. 15-A, for preloading the control gripper 113 with respect to the support 111 for the extraction.

The longitudinal axis X-X of the control gripper 113 can in some operating configurations not coincide with the longitudinal axis of the guide 131 of the handle 111.

The inclusion of the spherical joint 123 allows also controlling the pitch degree of freedom of the master device 110 with the fingers F1, F2 of the hand H of the surgeon 150 instead of controlling it with the wrist/elbow.

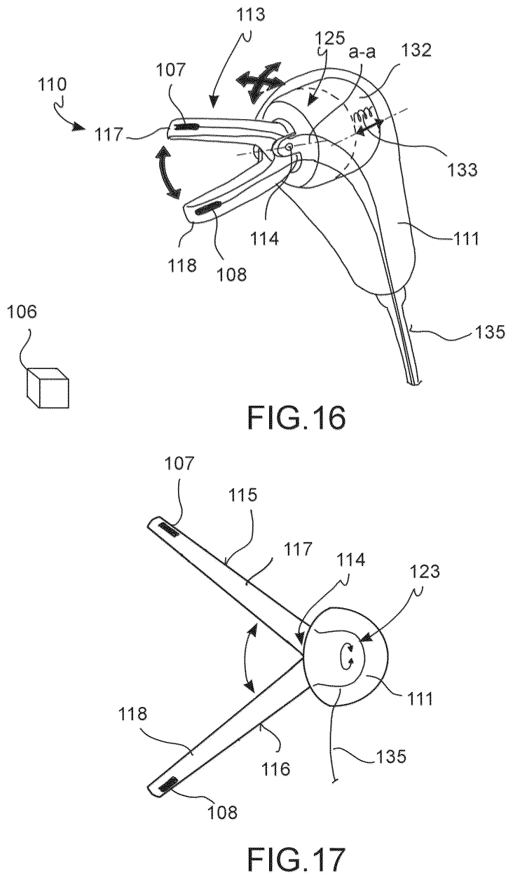

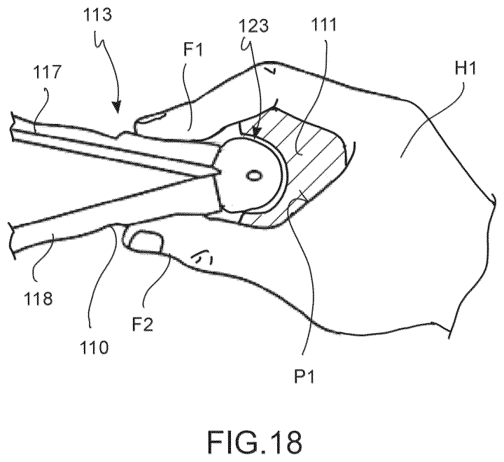

As shown for example in FIG. 16, where included, cables such as data connection cables 135 of the identifiers 107, 108, for example sensors 107, 108, can pass through the body of the control gripper 113 and through a passage provided inside the body of the handle 111. Therefore, the master workstation 101 comprising said reference 106 is capable, in accordance with an embodiment, of detecting information on the relative opening closing movements closing in a plane (for example distance and/or angle between the sensors 107, 108), longitudinal movement (for example forward backward with respect to the palm P1 of the hand H1 of the surgeon 150), and three-dimensional rotations.

As shown for example in FIG. 17, the handle 111 or support 111 can be cap-shaped, facing a domed or capped surface at the palm P1 of the hand H1 of the surgeon 150. The joint 114 between the rigid parts 117, 118 can be formed by an elastically flexible portion of the rigid parts themselves.

The range-of-motion of the joint between handle 111 or support 111 and control gripper 113 can be preset by providing mechanical end-of-strokes in or near the joint itself, as well as alternatively or in addition by including preloading elastic elements. The extension of said range of motion can depend on ergonomic handling reasons and can be adjusted during the design and/or assembly step of the master controller device.

In accordance with an embodiment, the joint between handle 111 or support 111 and control gripper 113 allows a roll rotation less than 360°, and for example belonging to the range +/−160° with respect to a median reference value. In accordance with a preferred embodiment, the joint between handle 111 or support 111 and control gripper 113 allows a roll rotation less than 180°, and for example belonging to the range +/−80° with respect to a median reference value.

In accordance with an embodiment, the joint between handle 111 or support 111 and control gripper 113 allows a longitudinal translation less than 2 cm, and for example allows about 1 cm of retraction of the control gripper 113 with respect to the support 111 due to the manipulation by the fingers F1, F2 of the hand H of the surgeon 150. The retraction can impose counteracting the action of a spring 133.

In accordance with an embodiment, the joint between handle 111 or support 111 and control gripper 113 allows a yaw rotation having a range-of-motion of about 90°, preferably belonging to the range +/−45° with respect to a median reference position. In accordance with an embodiment, the joint allows a yaw rotation having a range-of-motion of about 60°, preferably belonging to the range +/−30° with respect to a median reference position.

In accordance with an embodiment, the joint between handle 111 or support 111 and control gripper 113 allows a pitch rotation having a range-of-motion of about 90°, preferably belonging to the range +/−45° with respect to a median reference position. In accordance with an embodiment, the joint allows a pitch rotation having a range-of-motion of about 60°, preferably belonging to the range +/−30° with respect to a median reference position.

In accordance with a general embodiment, an ungrounded master controller device 110 is provided, which comprises a support 111 comprising a surface 112 for the palm of a surgeon's hand, and a control gripper 113 mounted to the support 111 and comprising two opposite manipulation interfaces 115, 116 for the fingers of said surgeon's hand; and in which the control gripper 113 comprises a first rigid part 117 and a second rigid part 118, placed side by side; said first rigid part 117 is constrained to rotate with respect to the support 111 about a first axis; said second rigid part 118 is constrained to rotate with respect to the support 111 about a second axis, coincident with or parallel to said first axis; the master controller device 110 can be associated with a tracking system configured to detect information on position and orientation of the first rigid part 117 and the second rigid part 118 of the control gripper 113, individually, to control a slave device 170 of the robotic system 100.

In accordance with an embodiment, the master controller device 110 is according to any one of the previously described embodiments.

A method of controlling a robotic system for medical or surgical teleoperation will be described below.

The control method comprises the step of providing a system for medical or surgical teleoperation comprising a master workstation 101, according to any one of the previously described embodiments, and a slave device comprising at least one slave surgical instrument 170. The slave surgical instrument 170 is preferably according to any one of the previously described embodiments.

The method comprises the step of controlling with the fingers F1, F2 of a hand H1 both the open/close degree of freedom and the yaw degree of freedom of the slave surgical instrument. The fingers are preferably index finger and thumb.

In accordance with a preferred embodiment, the method further comprises controlling the pitch degree of freedom with the same fingers of the surgeon's hand.

In accordance with a preferred embodiment, the method further comprises controlling the roll degree of freedom with the same fingers of the surgeon's hand.

By virtue of the features described above, given either separately or in combination, where applicable, it is possible to respond to the needs mentioned above, and to obtain the listed advantages, in particular:
it is allowed controlling, with the fingers F1, F2 of the hand H of the surgeon 150 with the palm P1 on the surface 112 of the handle or support 111, a control gripper 113 for surgical teleoperation in a plurality of degrees of freedom thereof;
the control gripper is a sensorized control gripper intended to be read by the tracking system while the handle is transparent to the tracking system and performs an ergonomic function;
the inclusion of redundant joints of the master controller device forming internal degrees of freedom of the master controller device adapted to transmit movements, by detecting the identifiers of the control gripper, to a slave surgical instrument allows the surgeon to amplify the control over the slave surgical instrument with only his fingers F1, F2, for example index finger and thumb of the hand grasping the master device;
the use of the fingers F1, F2 allows a finer and more precise and less tiring control for the surgeon, particularly in the case of a master device freely movable in the workspace (ungrounded or steering wheel-type master);
in particular, as compared with the use of the wrist or elbow or shoulder, the use of the fingers F1, F2 of the hand for the manipulation of various degrees of freedom of the control gripper which translate into the manipulation of enslaved degrees of freedom of the slave device, allows a more precise and therefore highly desirable surgical teleoperation;
it is thus allowed extending the range-of-motion of the surgeon's fingers, i.e., the workspace of the surgeon's fingers, by providing said one or more degrees of freedom inside the master device;
the movement of the rigid parts towards/away from each other preferably occurs in a definable plane, it thus allows activating, i.e., moving only the corresponding tips or jaws 171, 172 of the articulated end 175 of the slave device 170, while the movement of the yaw joints (which can coincide with the opening-closing joint between the jaws), pitch and roll of the slave surgical instrument 170 by moving the joint between handle or support and control gripper of the master device, all using only the fingers F1, F2 for example index finger and thumb of the hand H1 with the palm P1 on the surface 112 of the support or handle 111;
in particular, where a spherical joint is included between handle or support and control gripper, it is possible to control all the slave roll, pitch and yaw joints by using only the fingers F1, F2;
where a translation (retraction) degree of freedom is included, it is also possible to control with the fingers a linear translation degree of freedom of the positioning shaft 176 of the slave surgical instrument 170 (for example one or more linear slides, for example a robotic manipulator 160 of the Cartesian type);
no sensorized body is included between the rigid parts of the control gripper because the orientation is transmitted by virtue of the calculation of the control point between the identifiers or sensors, i.e., between the rigid parts;
by virtue of the suggested solutions, it allows accommodating the natural movements of the surgeon's hand maneuvering the master device, due to the kinematics of the human hand.
by virtue of the suggested solutions, it avoids transmitting unwanted control signals to the slave device and avoids, or at least minimizes, the risk of undesirable repositioning of the master device in the surgeon's hand.
by virtue of the suggested solutions, ergonomics and safety are improved with respect to known solutions.
by virtue of the suggested solutions, an intuitive, ergonomic, stable and fall-proof master device is provided.
by virtue of the suggested solutions, a master is provided consisting of a limited number of mechanical, electronic and sensor components and with an easily sterilizable or drapeable design with sterile cloth,
by virtue of the suggested solutions, a master controller device is provided which facilitates a controlled, precise and accurate movement and expands the range of motion of the different relative degrees of freedom and in particular the yaw degree and roll degree.
by virtue of the suggested solutions, a master device is provided consisting of a limited number of mechanical, electronic and sensor components, and/or simple ergonomics, and/or a solid fall-proof grip which however does not compromise the relative freedom of movement and the range of motion and/or which has an easily sterilizable design with sterile cloth, and/or facilitates a controlled, precise and accurate movement also and in particular on the yaw and roll degree.

In order to meet specific, contingent needs, those skilled in the art may make several changes and adaptations to the above-described embodiments and may replace elements with others which are functionally equivalent, without departing from the scope of the appended claims.

### LIST OF REFERENCE SIGNS

100 Robotic system for medical or surgical teleoperation
101 Master workstation assembly
106 Tracking device reference
107 First rigid part identifier
108 Second rigid part identifier
110 Master controller device, or master device
111 Handle or support
112 Surface for the palm
113 Control gripper
114 Control gripper joint
114′ Further control gripper joint
115 Manipulation interface of the first rigid part
116 Manipulation interface of the second rigid part
117 First rigid part, or rod
118 Second rigid part, or rod
119 Control gripper hub
120 Vision system
121 Cylindrical roll joint
123 Spherical joint
124 Cylindrical yaw joint
126 Elastic element of the control gripper
127 Fee end of the first rigid part
128 Free end of the second rigid part
131 Hole or seat or cavity or guide of the support for the translation of the control gripper
132 Cylindrical surface of the control gripper
133 Joint spring
134 Joint connecting rod
150 Surgeon
160 Robotic manipulator
170 Slave surgical instrument, or slave device
171 First tip link, or first jaw
172 Second tip link, or second jaw
173 Support link
174 Further proximal link
175 Slave articulated end
176 Positioning rod or shaft
177 Tendon
178 Free end
179 Slave control point
F1 Surgeon's hand finger, e.g., index finger
F2 Surgeon's hand finger, e.g., thumb
H1 Surgeon's hand
P1 Surgeon's hand palm
X-X Longitudinal axis of the control gripper
Y-Y Handle or support extension axis
Y Yaw rotation axis of the slave surgical instrument
R Roll rotation axis of the slave surgical instrument
P Pitch rotation axis of the slave surgical instrument
α Handle angle
β Angular stroke of the control gripper

1. A master workstation assembly for a robotic system for medical or surgical teleoperation, comprising:
at least one ungrounded master controller device, adapted to control at least one open/close degree of freedom and one yaw degree of freedom of a slave surgical instrument of the robotic system,
a tracking system to detect information on position and orientation of the master controller device;
wherein the master controller device comprises:
a handle comprising a surface for a palm of a surgeon's hand;
a control gripper coupled to the handle and comprising two opposite manipulation interfaces for fingers of said surgeon's hand;
and wherein
the control gripper comprises a first rigid part and a second rigid part, placed side by side;
said first rigid part is constrained to rotate with respect to the handle about a first axis;
said second rigid part is constrained to rotate with respect to the handle about a second axis, coincident with or parallel to said first axis;
said tracking system is configured to detect information on position and orientation of the first rigid part and the second rigid part of the control gripper, individually.

2. The assembly according to claim 1, wherein the control gripper comprises an elastic element between the first rigid part and the second rigid part biasing the first rigid part and the second rigid part away from each other.

3. The assembly according to claim 1, wherein the tracking system is configured to process, based on the detected information on position and orientation of the first rigid part and the second rigid part individually, a control point containing information on position and orientation of the control gripper.

4. The assembly according to claim 1, wherein the tracking system comprises:
a reference;
two identifiers fixed to the respective rigid parts of the control gripper, to detect position and orientation of each rigid part with respect to the reference.

5. The assembly according to claim 4, wherein the tracking system comprises:
two tracking sensors, each fixed to a respective rigid part of the control gripper, acting as identifiers; and
a tracking field generator, acting as said reference.

6. The assembly according to claim 4, wherein the tracking system comprises:
two optical markers, each fixed to a respective rigid part of the control gripper, acting as identifiers; and
an optical sensor, acting as a reference.

7. The assembly according to claim 4, wherein the tracking system comprises:
two encoders mounted to the handle and each operatively associated with a respective rigid part of the control gripper.

8. The assembly according to claim 1, wherein each rigid part of the control gripper is fixed in a rotational joint thereof to the handle, individually.

9. The assembly according to claim 1, wherein the control gripper comprises a rotational joint thereof between the two rigid parts constraining the two rigid parts to rotate about a common axis coinciding with said first axis.

10. The assembly according to claim 1, wherein the master controller device comprises a rotational joint between the handle and the control gripper.

11. The assembly according to claim 10, wherein the rotational joint between the handle and the control gripper comprises a cylindrical yaw joint with an axis parallel to the at least one joint between the rigid parts and a connecting rod for connection with the at least one joint between the rigid parts.

12. The assembly according to claim 10, wherein the rotational joint between the handle and the control gripper comprises a cylindrical roll joint which allows roll rotation of the control gripper with respect to the handle.

13. The assembly according to claim 10, wherein the rotational joint between the handle and the control gripper comprises a spherical joint.

14. The assembly according to claim 10, wherein the rotational joint between the handle and the control gripper further comprises an elastic element for biasing the rotational joint in a predeterminable orientation.

15. The assembly according to claim 10, wherein the rotational joint between the handle and the control gripper comprises a guide which allows the translation of the control gripper with respect to the handle.

16. The assembly according to claim 15, comprising a spring for biasing the control gripper for extraction with respect to the handle.

17. The assembly according to claim 1, wherein the handle is transparent to the tracking system, acting as an ergonomic component to impose positioning of the surgeon's fingers on the control gripper.

18. A method of controlling a robotic system for medical or surgical teleoperation comprising the steps of:
providing a system for medical or surgical teleoperation comprising a master workstation assembly according to claim 1, and a slave device comprising at least one slave surgical instrument;
controlling by fingers of a hand both the open/close degree of freedom and the yaw degree of freedom of the slave surgical instrument.

19. The assembly according to claim 1, wherein the tracking system is configured to process, based on the detected information on position and orientation of the first rigid part and the second rigid part individually, a control point containing information on position and orientation of the control gripper; and wherein the control point is calculated as a midpoint between the rigid parts of the control gripper.

20. The assembly according to claim 1, wherein the handle is transparent to the tracking system, and has no sensors and/or identifiers, acting as an ergonomic component to impose positioning of the surgeon's fingers on the control gripper.
